(19) United States
(12) Patent Application Publication
(10) Pub. No.: US 2013/0287757 A1
(43) Pub. Date: Oct. 31, 2013

(54) COMPOSITION FOR THE REGENERATION OF ATROPHIC TISSUES

(71) Applicants: Luisa Gennero, Torino (IT); Fulvio Pedrini, Torino (IT); Maurizio Pedrini, Torino (IT); Gianfranco Merizzi, Torino (IT)

(72) Inventors: Luisa Gennero, Torino (IT); Fulvio Pedrini, Torino (IT); Maurizio Pedrini, Torino (IT); Gianfranco Merizzi, Torino (IT)

(73) Assignee: MEDESTEA INTERNAZIONALE S.P.A., Torino (IT)

(21) Appl. No.: 13/979,581

(22) PCT Filed: Jan. 13, 2011
(86) PCT No.: PCT/IB2011/050153

§ 371 Date: Jul. 12, 2013

(51) Int. Cl. A61K 31/19; A61K 31/198; A61K 33/30; A61K 31/60; A61K 38/48

(52) U.S. Cl. CPC A61K 31/19; A61K 31/60; A61K 38/4873; A61K 33/30; A61K 31/198
USPC 424/94.65; 435/404

(57) ABSTRACT

An effective composition is for the therapeutic or cosmetic treatment of atrophic cutaneous and subcutaneous tissues, skin appendages, connective tissues or mucous tissues. The composition includes a combination of a salt of adamantane carboxylic acid, preferably salified with an aromatic and/or adamantane base, with a proteolytic enzyme and/or willow extract, salicylic acid, acetylsalicylic acid, salicylate, or acetylsalicylate. Further active ingredients that are suitable for inclusion in the composition are zinc oxide, vitamins, phytoextracts, sugars, amino acids, minerals, further salts, phytoextracts, ventilated green clay and any combination thereof. The composition has a particular synergy between the constituent elements, and is particularly effective in the treatment of atrophic ulcers or bedsores.

The present invention relates to a composition that is effective in the regeneration of atrophic tissues, particularly ulcerated tissues with atrophy and loss of tissue, of locoregional vascularization and of locoregional innervation, and possibly with necrosis. The atrophic and/or ulcerated and/or necrotic tissue is in particular a cutaneous tissue, a skin appendage, a mucous tissue or a connective tissue.

The composition of the invention lends itself to be prepared in various physical forms and formulations specifically suited to the intended use, such as cosmetic compositions, cell culture media, pharmaceutical compositions, and medical devices, for human or veterinary use.

As will be illustrated in detail in the section relating to the examples, the composition of the invention is effective in the regeneration and in the eutrophic restoration of atrophic or atrophic-necrotic cutaneous tissues, skin appendages, mucous tissues and connective tissues, both in vitro and in vivo, creating the micro-environmental conditions suitable for inducing tissue regeneration by stimulating loco-resident native stem cells to differentiate spontaneously with the synthesis and physiological secretion of collagen.

The expression “skin appendages” means the sweat glands, hair follicles, hairs, sebaceous glands and the muscle of the hair in animals.

The term “ulcer” generally means a wound that does not tend to heal spontaneously.

The most frequent types of ulcer are those due to chronic venous insufficiency, the so-called “venous ulcers”. They are called “varicose ulcers” when the venous insufficiency is superficial, due to the presence of varicose veins. The term ulcer further comprises the so-called “bedsores” and “atrophic ulcers”, i.e. lesions that occur when the compressing force, applied for a long enough time, is greater than the blood pressure in the arteriolocapillary compartment, so as to cause ischaemia.

### SUMMARY OF THE INVENTION

The purpose of the present invention is to provide a composition of active ingredients that is effective in the treatment of atrophic tissues, possibly ulcerated and/or necrotic, in vitro and in vivo, in humans and animals, in particular atrophic cutaneous tissue, mucous tissue and connective tissues.

This purpose is achieved by means of the composition of active ingredients defined in the appended claims, which form an integral part of the present description.

The inventors found that the composition of the invention is effective, synergistically between its various constituents, in inducing, both in vitro and in vivo, the repair and regeneration of tissues that are atrophic, possibly ulcerated and/or necrotic, such as cutaneous tissue, mucous tissue and connective tissues with atrophic ulcers, by restoring physiological, biochemical and micro-environmental conditions that are optimum for stimulating the vitality and improving the trophism of the compromised tissues. More particularly, the inventors found that the composition of the invention is effective in treatment against ulcers, bedsores and in particular in anti-necrotizing and/or reparative-regenerative infralesional and perilesional treatment of vascular tissue and/or peripheral nerve tissue and/or of the integumentary system and/or of the perilesional connective tissue.

The composition of the invention is based on a combination of one or more salts of adamantane carboxylic acid (preferably adamantane carboxylic acid salified with at least one amine selected from the group comprising adamantane and/or aromatic amines as described below) and with at least one further active ingredient selected from (i) proteolytic enzymes (preferably papain) and (ii) salicylic acid, acetylsalicylic acid, salts derived therefrom (salicylate or acetylsalicylate) or willow extract.

The terms “salicylic acid” and “acetylsalicylic acid” include said substances in any form, for example salicylic acid F.U. and acetylsalicylic acid F.U. The same applies to the salicylates and acetylsalicylates.

The term “salt of adamantane carboxylic acid” means both the salts of adamantane monocarboxylic acid and the salts of adamantane dicarboxylic acid.

Other ingredients suitable for use in the composition of the invention are zinc oxide, vitamins, phytoextracts (preferably glycyrrhetic acid), sugars, amino acids, minerals and other salts.

Depending on the intended applications and uses, the composition of the invention can be supplied as a pharmaceutical composition (in various tissue-specific formulations), a medical device, a cosmetic composition or as a means of cell culture for use in vitro.

As will be described in greater detail below, the composition of the invention was tested both in vitro and in vivo. The results obtained confirm its efficacy in the repair, regeneration and retrophization of atrophic tissues, including ulcerated and/or necrotic tissues, such as dermis, mucosae, hypodermis and connective tissue in general. Particularly significant histological results were obtained in vitro after six months of treatment of biopsies of atrophic scars, demonstrating the synergistic action of the constituents of the new composition. The tissue repair and retrophization induced by the composition of the invention is morphologically comparable to the trophic condition of intact tissues in vivo, with optimum histo-functional characteristics. It should be pointed out that with the normal media for culture in vitro, tissues from skin biopsies do not generally live longer than a month.

### DETAILED DESCRIPTION OF THE INVENTION

The present invention makes available a composition that is valid and effective in the treatment of atrophic tissues, including ulcerated and/or necrotic tissues, so as to permit the correct synthesis, metabolism and catabolism of collagen and to speed up tissue repair. In particular, the present invention makes available a solution that is valid and effective for improving the vitality and trophism of epidermal, dermal, and mucous tissue and connective tissue in general, when compromised by atrophy, including ulcers and/or necrosis, by creating suitable conditions to promote the repair, regeneration and induction to intra-tissue differentiation of pluripotent stem cells present in the tissues in question, native and non-native.

The invention is based on the surprising observation of a particular pro-proliferative stimulus exerted by the combination of active ingredients described above.

The present inventors in fact found, surprisingly, that this combination of active ingredients has a dual action, on the one hand antimicrobial and on the other hand regulation of the metabolism and catabolism of collagen, which promotes eutrophism of cutaneous and connective tissue, correct locoregional vascular and nerve regeneration and balanced cellular proliferation (fibroblasts etc.).

Moreover, the present inventors found that the proteolytic enzyme in combination with the salt of adamantane carboxylic acid and salicylic acid, acetylsalicylic acid or a salt thereof (salicylate or acetylsalicylate) or with willow extract, promotes the following activity: (i) deep locoregional bactericidal, virucidal, fungicidal and sporicidal action, (ii) activation of the growth factors present physiologically in the micro-environment present in damaged or senescent tissues, and (iii) activation of the same growth factors attracted by salicylic acid, by acetylsalicylic or by a salt thereof (salicylate or acetylsalicylate) or by willow extract, with consequent chemotactic induction of the resident stem compartment and rapid reparative differentiation.

Therefore, in a preferred embodiment, the composition of the invention comprises a combination of (i) salt of adamantane carboxylic acid, (ii) salicylic acid, acetylsalicylic acid or a salt thereof (salicylate or acetylsalicylate) or willow extract and (iii) proteolytic enzyme.

In another preferred embodiment, the composition of the invention comprises a combination of (i) salt of adamantane carboxylic acid, (ii) salicylic acid, acetylsalicylic acid or a salt thereof (salicylate or acetylsalicylate) or willow extract, (iii) proteolytic enzyme and (iv) zinc oxide.

Further ingredients that can be present in the composition of the invention are vitamins, phytoextracts (for example glycyrrhetic acid), sugars, amino acids, minerals and other salts. These further ingredients are selected on the basis of their potentiating or synergistic activity with the active ingredients mentioned above, specifically for each type of tissue to be treated.

The preferred concentrations of the aforementioned active ingredients, if present, are as follows.

The salt of adamantane carboxylic acid is preferably present in the composition at a concentration in the range from 0.001 to 20 mg/kg, more preferably from 0.2 to 10 mg/kg.

Salicylic acid, derivative of salicylic acid or salt derived therefrom is preferably present in the composition at a concentration in the range from 0.1 to 10 g/kg, more preferably from 4 to 8 g/kg.

The proteolytic enzyme, preferably papain, is preferably present in the composition at a concentration in the range from 0.01 μg/L to 100 mg/L, more preferably from 10 mg/L to 80 mg/L.

Each vitamin is preferably present in the composition at a concentration in the range from 0.0001 to 200 mg/kg, more preferably from 0.005 to 50 mg/kg.

The phytoextract, which is preferably glycyrrhetic acid, is preferably present in the composition at a concentration in the range from 20 to 5000 mg/kg, more preferably from 200 to 2000 mg/kg.

Zinc oxide is preferably present in the composition at a concentration in the range from 0.01 g/kg to 10 g/kg, more preferably from 1 to 2 g/kg.

The histological results obtained in vitro after treatment with the composition of the invention confirm the synergistic induction of all of the constituents of regeneration and retrophization of dermis, skin appendages, hypodermis, mucous and connective tissue, which are found to be morphologically comparable to intact tissues in vivo and with optimum histofunctional characteristics, with an efficacy greater than the action exerted by the individual constituents of said composition (see Example 1).

Treatment of biopsied atrophic scar tissue in vitro with the composition of the invention results in surprising repair of damage and in complete recovery of the original trophism. All the tissues represented in treated biopsy samples undergo complete functional and morphologic regeneration.

The histological results obtained in vivo starting from 30-60 days of treatment with the composition according to the present invention and its tissue-specific formulations (see the section relating to clinical studies) confirm, on histologic examination, the development of regeneration and retrophization of dermis and hypodermis, mucosae and connective tissue in general with optimum histofunctional characteristics, morphologically comparable to intact cutaneous and subcutaneous, mucous and connective tissues in vivo, with greater efficacy relative to the action exerted by the individual constituents of said composition.

Tables 1 to 9 given below illustrate some specific examples of compositions according to the invention. These compositions differ from one another with respect to the dosage form (cream, gel, liquid, liquid for infusion, etc.) and based on the type of tissue for which they are specifically indicated. The compositions in Tables 1 to 9 can be modified as indicated in the course of the description without leaving the scope of the invention, and obtaining similar results in terms of efficacy.

For example, as an alternative to the 1:1 mixture of 1-adamantane acetate of N,N-dimethyl-phenylethylammonium and 1,3-adamantane dicarboxylate of bis(1-adamantane ammonium), it is possible to use the same amount of 1-adamantane acetate of N,N-dimethyl-phenylethylammonium salt on its own or the same amount of 1,3-adamantane dicarboxylate of bis(1-adamantane ammonium) salt on its own.

As an alternative to papain it is possible to use any other proteolytic enzyme selected from the group comprising collagenase, serratiopeptidase, heparanase, elastase, bromelain, bradykinase, Clostridium peptidase, proteolytic enzymes expressed by Lactobacillus acidophilus, proteolytic enzymes expressed by the genus Aspergillus, protease, alliinase and fibrinolysin. The amount of proteolytic enzyme to use depends mainly on its specific activity, and determination thereof is within the ability of a person skilled in the art.

As an alternative to salicylic acid it is possible to use for example acetylsalicylic acid. Both salicylic acid and acetylsalicylic acid can be used in acid or salified form.

As an alternative to glycyrrhetic acid it is possible for example to use glycyrrhizin.

Making these substitutions, the inventors obtained results that are substantially equivalent, in terms of efficacy, to those obtained using the specific compositions in Tables 1-3. The specific compositions in Tables 1-3, which represent illustrative but non-limiting examples, displayed mutually potentiating and synergistic activity, specifically for each type of tissue to be treated. The efficacy of the tissue-specific compositions was always found to be significantly higher than the action exerted by the individual constituents of said compositions (complete compositions described in Tables 1-3 versus individual active constituents described in Tables 4-11).

TABLE 1
Composition UL-MD for the treatment of cutaneous and
subcutaneous tissue in vivo and in vitro

| | |
|---|---|
| | Concentration |
| Substance | mg/kg |

| | | |
|---|---|---|
| Active ingredients | | |
| 1:1 mixture of 1-adamantane acetate of N,N- | 200 | mL (from 2.5 |
| dimethyl-phenylethylammonium and 1,3- | | to 5 g/kg of |
| adamantane dicarboxylate of bis(1-adamantane | | active |
| ammonium) in solution | | ingredient in |
| | | total) |
| Salicylic acid | 8000 | mg/kg |
| Papain FU | 56.00 | mg/kg |
| Further active ingredients | | |
| Glycyrrhetic acid | 2000 | mg/kg |
| Excipients | | |
| Salts | 1 | g/kg |
| In vivo | | |

| | |
|---|---|
| BASE of gel (carbopol or cellulose derivatives), | q.s. |
| or | per kg of product, |
| BASE in the form of cream or emulsion (O/W or | or |
| W/O) (such as: water, white petroleum jelly, | per L of solution |
| cetostearyl alcohol, liquid paraffin, ceteth-20, | |
| sodium phosphate, p-chloro-m-cresol, phosphoric | |
| acid), | |
| or | |
| Saline | |
| In vitro | |
| Culture medium DMEM-LG/F12 (1:1) | |

TABLE 2
Composition UL-PI for the treatment of ulcers with loss of
cutaneous and subcutaneous tissue

| | Concentration | |
|---|---|---|
| Substance | mg/kg | |
| Active ingredients | | |
| 1:1 mixture of 1-adamantane acetate of N,N- | 200 | mL (from 2.5 |
| dimethyl-phenylethylammonium and 1,3- | | to 5 g/kg of |
| adamantane dicarboxylate of bis(1- | | active |
| adamantane ammonium) in solution | | ingredient in |
| | | total) |
| Salicylic acid | 8000 | mg/kg |
| Papain FU | 56.00 | mg/kg |
| Further active ingredients | | |
| Glycyrrhetic acid | 2000 | mg/L |
| Mixture of L-amino acids | 820 | mg/kg |
| Phytoextracts | 5 | g/kg |
| Excipients | | |

| | |
|---|---|
| BASE of gel (carbopol or cellulose derivatives), | q.s. |
| or | per kg of product, |
| BASE in the form of cream or emulsion (O/W or | or |
| W/O) (such as: water, white petroleum jelly, | per L of solution |
| cetostearyl alcohol, liquid paraffin, ceteth-20, | |
| sodium phosphate, p-chloro-m-cresol, | |
| phosphoric acid), | |
| or | |
| Saline | |

TABLE 3
Composition RITROFIX for the regenerative treatment of
cutaneous and subcutaneous atrophic lesions

| | Concentration | |
|---|---|---|
| Substance | mg/kg | |
| Active ingredients | | |
| 1:1 mixture of 1-adamantane acetate of N,N- | 200 | mL (from |
| dimethyl-phenylethylammonium and 1,3- | | 2.5 to 5 g/kg |
| adamantane dicarboxylate of bis(1- | | of active |
| adamantane ammonium) in solution | | ingredient |
| | | in total) |
| Salicylic acid | 8000 | mg/kg |
| Papain FU | 56.00 | mg/kg |
| Further active ingredients | | |
| Zinc oxide powder | 1100 | mg/kg |
| Glycyrrhetic acid | 2000 | mg/L |
| Mixture of L-amino acids | 820 | mg/kg |
| Vitamins | 112.574 | mg/kg |
| Phytoextracts | 110 | mg/kg |
| Ventilated green clay | 500 | mg/kg |
| Excipients | | |

| | |
|---|---|
| BASE of gel (carbopol or cellulose derivatives), | q.s. |
| or | per kg of product, |
| BASE in the form of cream or emulsion (O/W | or |
| W/O) (such as: water, white petroleum jelly, | per L of solution |
| cetostearyl alcohol, liquid paraffin, ceteth-20, or | |
| sodium phosphate, p-chloro-m-cresol, | |
| phosphoric acid), | |
| or | |
| Saline | |

TABLE 4
Adamantane active constituent

| Substance | Concentration |
|---|---|
| Active ingredients | mg/kg |
| 1:1 mixture of 1-adamantane acetate of N,N- | 200 mL (from 2.5 to 5 g/kg |
| dimethyl-phenylethylammonium and 1,3- | of active ingredient in total) |
| adamantane dicarboxylate of bis(1- | |
| adamantane ammonium) in solution | |

TABLE 5
Salicylate active constituent

| Substance | Concentration |
|---|---|
| Active ingredients | mg/kg |
| Salicylic acid | 8000 mg/kg |

TABLE 6
Proteolytic enzyme active constituent

| Substance | Concentration |
|---|---|
| Active ingredients | mg/kg |
| Papain FU | 56.00 mg/kg |

TABLE 7
Zinc oxide active constituent

| Substance | Concentration |
|---|---|
| Further active ingredients | mg/kg |
| Zinc oxide powder | 1100 mg/kg |

TABLE 8
Glycyrrhetic acid active constituent

| Substance | Concentration |
|---|---|
| Further active ingredients | mg/kg |
| Glycyrrhetic acid | 2000 mg/L |

TABLE 9
Amino acid active constituent

| Substance | Concentration |
|---|---|
| Further active ingredients | mg/kg |
| Mixture of L-amino acids | 820 mg/kg |

TABLE 10
Vitamin active constituent

| Substance | Concentration |
|---|---|
| Further active ingredients | mg/kg |
| Vitamins | 112.574 mg/kg |

TABLE 11
Active constituent in phytoextracts

| Substance | Concentration |
|---|---|
| Further active ingredients | mg/kg |
| Phytoextracts | 110 mg/kg |

### Composition Rationale

The present invention is based on observation of a particular reparative and regenerative stimulus exerted on dermis, mucosae, hypodermis and on the connective tissues by a combination of salts of adamantane carboxylic acids with (i) salicylic acid, acetylsalicylic acid or a salt thereof, and/or (ii) proteolytic enzyme, optionally in combination with one or more further active ingredients selected from zinc salts, amino acids, vitamins, other salts, phytoextracts, minerals. The composition of the invention can also contain further ingredients selected on the basis of the dosage form and the specific medical indication envisaged, for example mucopolysaccharides, antibiotics, amino-amide anaesthetics, parasympathicolytics, essential oils, ventilated green clay, etc.

### Adamantane Carboxylic Acid Salts

The salts of adamantane carboxylic acid that are preferred for use in the composition of the invention are salts of adamantane monocarboxylic or adamantane dicarboxylic acid with one or more adamantane bases and/one or more aromatic bases. Preferred examples of said salts are represented by the following general formula:

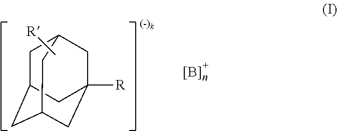

in which:
k=1 or 2;
n=1 or 2;
R is selected from R═—C(O)O— and -Ak-C(O)O⁻, in which Ak is optionally substituted C₁₋₆ alkyl;
R′ is selected from hydrogen, —C(O)O⁻ and -Ak-C(O)O⁻, in which Ak is optionally substituted C₁₋₆ alkyl (preferably methyl or ethyl); and
B is a counter-ion selected n times independently of one another from the group comprising bases of formulae (i) and (ii):

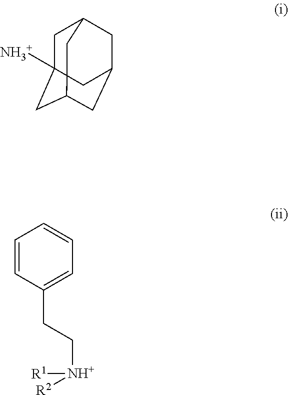

in which in formula (II) R¹ and R² are selected independently of one another between hydrogen and C₁₋₆ alkyl (preferably methyl or ethyl).

The expression “C₁₋₆ alkyl” includes the alkyl radicals of all the linear or branched alkyls having from 1 to 6 carbon atoms; among these, methyl and ethyl are particularly preferred.

Some salts of adamantane carboxylic acid covered by the general formula given above are described in Italian patent IT 1360272. Among these, we may mention in particular 1-adamantane acetate of N,N-dimethyl-phenylethylammonium and 1,3-adamantane dicarboxylate of bis(1-adamantane ammonium), having the following respective structural formulae:

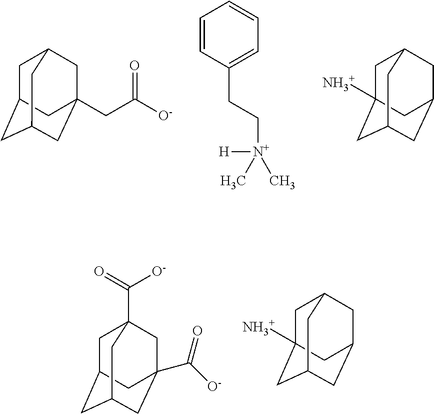

In patent IT 1360272, the salts of adamantane carboxylic acid and adamantane and/or aromatic bases are described as antimicrobial, antibacterial, and antiviral agents suitable for use in disinfectants for surfaces in hospitals, in industry, etc. The inventors established experimentally that these salts on their own do not possess any therapeutic effect or otherwise regenerative or reparative effect on atrophic tissues, particularly atrophic ulcers.

### Proteolytic Enzymes

The proteolytic enzyme that is preferred for use in the composition of the invention is papain.

However, other proteolytic enzymes can be used in place of papain, obtaining substantially the same results. Among the proteolytic enzymes that can be used in place of papain in the composition of the invention, we may mention as examples: collagenase (preferably of type Ia, of type II or of type IV), serratiopeptidase, heparanase, elastase, bromelain, bradykinase, Clostridium peptidase, proteolytic enzymes expressed by Lactobacillus acidophilus, proteolytic enzymes expressed by the genus Aspergillus, protease, alliinase, fibrinolysin.

### Salicylic Acid or Salicylates

Salicylic acid is preferably used in the composition of the invention in the form of salt. As an alternative to salicylic acid it is possible to use, obtaining substantially the same results, a derivative of salicylic acid, for example acetylsalicylic acid, or a willow extract.

### Zinc Oxide

It was established by the present inventors that zinc oxide, as well as having an emollient effect, is capable of inducing—synergistically with salicylic acid and the proteolytic enzyme—an increase in local vascularization that proves beneficial in atrophic and/or ulcerous and/or necrotic scars.

### Synergistic Potentiation of the Regenerative Action

Furthermore, it has been observed that salicylic acid, the proteolytic enzyme and zinc oxide perform various synergistic functions between them: (i) there is micro-dissection and superficial exfoliation of wounds, eliminating severely damaged cells and correcting the peripheral dysaemia that develops in the dermis after an injury, (ii) absorption of the nutrients present in the preparation is accelerated, and (iii) there is induction of tissue repair, formation of proteins, hormonal functions and processes of skin regeneration by inhibiting the lipases of bacteria, yeasts, and various saprophytes of the skin.

### Vitamins

Vitamins suitable for use in the composition according to the invention are for example retinoic acid, retinaldehyde, retinol, alpha-tocopherol, beta-carotene, colecalciferol, ascorbic acid, pantothenic acid, dexpanthenol, D-calcium pantothenate, cocarboxylase tetrahydrate, pyridoxine, pyridoxine-HCl, folic acid, niacinamide, nicotinamide, riboflavin, riboflavin dihydrate sodium phosphate, cyanocobalamin, para-aminobenzoic acid, biotin.

The inventors found that vitamin A, used at doses without any pharmacologic effect, potentiates correct nutrition of the tissues, leading to gradual physiological restoration of the micro-environment of the dermis and hypodermis (restoration of the correct pH and antioxidant power).

Still on the basis of experiments performed by the present inventors, it was established that within the scope of the composition according to the present invention, the group B vitamins, preferably B1, B2, B5, B6, B9 and B12, contribute to normalization of the locoregional micro-environment, promoting ideal exchange of albumin and of the cell nuclei promoting correction of the protein and electrolyte concentration that was altered in the course of hypertrophic tissue reactions.

Furthermore, it was found that within the scope of the composition according to the present invention, vitamin C promotes restoration of physiological synthesis of collagen in the damaged dermis and in the basement membrane, while vitamin D at physiological values performs an auxiliary role in consolidation of ideal sodium-calcium ion exchange promoting correction of the altered electrolyte concentration to physiological values.

The inventors further observed that vitamin E, used at doses without any pharmacologic effect, leads to gradual physiological restoration of the micro-environment, especially subcutaneous, actively opposing imperfections resulting from hypertrophic tissue changes, and assists the physiological biosynthesis of collagen by the locoregional fibroblasts.

In relation to the activity of vitamin H (biotin) within the composition according to the present invention, it was found that this molecule induces a locoregional anti-inflammatory and anti-oedematogenic action, restoring reduced permeability of the physiological micro-environment and promoting activation of the body's two repair mechanisms: attraction of stem cells from the bloodstream and accelerated specialization of stem cells residing in the skin towards rapid differentiation into mature cells intended to restore the damaged tissues.

Moreover, the present inventors found that vitamin PP (nicotinamide), in combination with the various components of the composition according to the present invention, induces various mechanisms of action locoregionally:
1) it performs functions as a constituent of enzymes involved in redox reactions;
2) it is involved in the energy metabolism of polysaccharides and proteins;
3) it forms part of the ferments of the capillary metabolism;
4) it is involved, as active cofactor for the skin, in the synthesis of sex hormones.

### Ventilated Green Clay

With regard to Ventilated Green Clay, the present inventors established that it has excellent absorbent and thickening power when it is present in gel or cream formulations; moreover, it has good remineralizing capacity and activates cation exchange in tissues.

### Amino Acids

The composition according to the present invention preferably comprises one or more amino acids or combinations of amino acids.

In a preferred embodiment, the composition of the invention comprises a combination of L-glycine, L-leucine, L-lysine, L-proline and optionally other amino acids. These amino acids constitute eutrophizing elements that function as important growth factors for the skin (basal layer) and the hypodermis, regulating cell and tissue turnover.

Among the further amino acids that can be present in the compositions of the invention, we may mention as examples: methionine, cystine, N-acetylcysteine, cysteine, isoleucine, glutamine, arginine, glutamic acid, histidine, histidine-HCl, lysine-HCl, phenylalanine, serine, threonine, tryptophan, tyrosine, tyrosine-disodium salt, valine, hydroxyproline. In addition or as an alternative to the amino acids, the composition of the invention can comprise peptides and/or proteins, for example glutathione, glycocholic acid, soya lecithin, collagen, elastin, wheat extract, and the like.

### Phytoextracts

Vegetable extracts suitable for use in a composition according to the invention are for example: glycyrrhizin, glycyrrhetic acid, liquorice extracts, aloe extracts, aloe vera extracts, pineapple extracts, mallow extracts, extracts of St John's Wort, burdock extracts, lime extracts, borage extracts, clover extracts, mint extracts, juniper extracts, lavender extracts, marigold extracts, extracts of horse chestnut, grapefruit extracts, cedar extracts, myrtle extracts, pine extracts. Essential oils suitable for use in a composition according to the invention are for example oil of St John's Wort, borage, mint, juniper, lavender, marigold, horse chestnut, grapefruit, cedar, pine, myrrh, incense, amber.

### Salts

Salts suitable for preservative use of a composition according to the invention are for example calcium gluconate, calcium phosphate, sodium bicarbonate, calcium chloride, magnesium chloride, magnesium sulphate, potassium chloride, potassium phosphate, sodium chloride, calcium nitrate, zinc chloride, ferric nitrate, sodium pyruvate, D-calcium pantothenate, tyrosine disodium salt. Minerals, in the form of salts, suitable for use in a composition according to the invention are for example silicates, carbonates, halides, oxides, sulphates, sulphides, phosphates, native elements such as silver, gold and copper. More preferably: aluminosilicates such as clay and ventilated green clay, carbonates including calcite and calcium carbonate and native elements such as micronized silver.

The cosmetic and pharmaceutical compositions and those of the medical device type according to the present invention can moreover comprise further subsidiary elements such as excipients and vehicles, the selection and use of which are within the capability of a person skilled in the art without his having to perform any inventive activity. In this connection and purely as a non-binding example, we may mention the mucopolysaccharides as suitable for making up the consistency of a composition according to the invention (hyaluronic acid, chondroitin sulphates, etc.).

The compositions of the invention prepared as culture media were tested to verify their effectiveness in preserving the vitality of atrophic biopsy samples. All the biopsy samples of atrophic scar tissue tested responded positively to the use of the culture media of the invention, remaining vital, reorganizing the three-dimensional tissue structure and depositing collagen physiologically with ordered, three-dimensional redistribution for at least six months of culture in vitro. Without wanting to be bound to any specific theory in this respect, the present inventors think that the results obtained with the culture media according to the present invention made it possible to demonstrate that the state of cutaneous atrophy taking place in degenerative processes is recoverable.

According to the present invention the culture media can comprise further ingredients, for example the usual inorganic salts, sugars, peptides, amino acids and vitamins necessary for the maintenance and/or growth in culture of mammalian cells, as well as optional antibiotics and/or antimicrobial agents necessary to avoid contamination of the cultures.

Examples of solutions for cell culture to which the composition according to the present invention can be added are for example RPMI 1640 (cell culture medium), DMEM-LG (cell culture medium), AIM-V (cell culture medium), modified D-MEM with high glucose concentration (cell culture medium), EBM (cell culture medium), human albumin, FBS (fetal bovine serum for cell cultures), F12 (solution for cell cultures containing a complete amino acid source), Hanks' solution (solution for cell cultures containing sodium bicarbonate).

The following examples describe tests performed in vitro on biopsies of atrophic tissues and the clinical tests conducted in vivo on animal and human subjects with compositions covered by the present invention. These examples are provided merely for purposes of illustration and do not limit the scope of the present invention as defined in the appended claims.

### Example 1

### Biopsies and Prototype Solutions

### Biopsies

The animal biopsy samples investigated are constituted of biopsies from chronic atrophic ulcers (base and margins).

All the samples were washed three times with saline and antibiotics (100 units/ml of penicillin+100 μg/ml streptomycin+160 mg/L gentamicin, fluconazole 0.2 mg/ml) for 10 min at room temperature.

The biopsies were then sectioned into twelve parts (two controls, 1-11, and one sample, 12, to be treated for each patient) and suspended in a final volume of 25 ml of the respective culture solutions in plates (Lab-Tek chamber slides, Nunc, Kamstrup, Denmark) of 10 cm.

Eleven types of control were prepared:
a negative control, untreated (1), i.e. treated exclusively with saline and antibiotics (as described above);
a positive control treated (2) with cell culture media commonly used for skin biopsies;
a control-3 treated with DMEM-LG/F 12 (1:1) culture medium and with the active ingredient described in Table 4.
a control-4 treated with DMEM-LG/F 12 (1:1) culture medium and with the active ingredient described in Table 5.
a control-5 treated with DMEM-LG/F 12 (1:1) culture medium and with the active ingredient described in Table 6.
a control-6 treated with DMEM-LG/F 12 (1:1) culture medium and with the active ingredient described in Table 7.
a control-8 treated with DMEM-LG/F 12 (1:1) culture medium and with the active ingredient described in Table 8.
a control-9 treated with DMEM-LG/F 12 (1:1) culture medium and with the active ingredient described in Table 9.
a control-10 treated with DMEM-LG/F12 (1:1) culture medium and with the active ingredient described in Table 10.
a control-11 treated with DMEM-LG/F12 (1:1) culture medium and with the active ingredient described in Table 11.
   One type of sample (12th portion of each biopsy investigated) was prepared, as follows:
sample-12: the biopsy findings of sample (12) were placed in plates (Lab-Tek chamber slides, Nunc, Kamstrup, Denmark) of 10 cm in culture medium in a solution Culture medium UL-MD.

### SUMMARY

1. Negative control: the biopsy findings of control 1 were suspended in saline in plates (Lab-Tek chamber slides, Nunc, Kamstrup, Denmark) of 10 cm.

2. Positive controls: the biopsy findings of control 2 were placed in plates (Lab-Tek chamber slides, Nunc, Kamstrup, Denmark) of 10 cm in D-MEM medium supplemented with:
10% FBS (Celbio, Milan, Italy)
160 mg/L gentamicin (Schering-Plough, Milan, Italy)
2 mM L-glutamine (Life Technologies; growth medium)
50 ng/mL EGF (Sigma-Aldrich, Milan, Italy).

3-11. Internal controls: the biopsy findings of control 3-11 were suspended in plates (Lab-Tek chamber slides, Nunc, Kamstrup, Denmark) of 10 cm, in DMEM-LG/F12 (1:1) culture medium and with the respective active ingredients described in Tables 4 to 11.

12. Samples: the biopsy findings of sample (12) were placed in plates (Lab-Tek chamber slides, Nunc, Kamstrup, Denmark) of 10 cm in culture medium in a solution Culture medium UL-MD.

All the findings were incubated for 15 days in a Heraeus incubator thermostatically controlled to a temperature of 37° C. with an atmosphere containing 5% of constant supply of CO₂ (v/v in air). Two thirds of the culture medium was replaced every 7 days. All biopsy tissues used in culture constitute a possible optional substrate co-conditioning for the three-dimensional growth of the cellular samples investigated.

### Staining Protocol

After three washings for 10 min at room temperature in PBS (pH 7.4), the samples were resuspended in a fixing solution of paraformaldehyde at 4% in D-MEM-LG (Gibco) at pH 7.4, for 1 hour at room temperature. All the biopsies under investigation were treated with Alcian blue. This dye is constituted of a group of water-soluble polyvalent basic colours. The blue colour is due to the presence of copper in the molecule. Alcian blue in solution with PBS (pH 7.4) at 1% of final concentration w/v is added to a 3% solution of acetic acid

(pH 2.5). This composition, after incubation for 2 hours at room temperature, stains indelibly by binding the acidic mucopolysaccharides and the glycoproteins, both sulphonates and carboxylates. Specific controls were prepared for each sample (controls 1-11). All the samples were washed 3 times with PBS (pH 7.4) at room temperature for five minutes and then examined with a light microscope.

A marked increase in collagen type 2 and collagen type 4, which stain blue, is noted in the samples treated with the solution Culture medium UL-MD relative to the controls (negative control 1, positive control 2, internal controls 3-11).

### Results

Coloration with the Colorimetric Method Alcian Blue

Negative Controls (1),

treated with saline: nonspecific blue coloration (score=+/−) alternating with cytolytic and necrotic areas;

Positive Controls (2),

treated with common culture medium for biopsies, as described above. A diffuse light blue coloration is noted (Alcian blue, score=++);

Internal Controls (3-11),

treated with DMEM-LG/F 12 (1:1) culture medium and with the respective individual active ingredients as described in Tables 4 to 11, as detailed above. A pale pericellular blue coloration is noted (Alcian blue, (score=+);

Samples—(12),

treated with the solution Culture medium UL-MD. It is noted that the cells in which a re-deposition of mucopolysaccharides and glycoproteins was induced are clearly stained with Alcian blue increasing in superposed layers arranged with physiological distribution of mucopolysaccharides and GAG (score=+++++).

### Western Blot (WB)

The samples and the controls underwent phenotypic analyses by Western Blot for the markers (Santa Cruz Biotechnology, California, USA) anti-collagen type I, anti-collagen type IV, anti-cytokeratins 1, 5, 10, 14. After five washings, the membranes were incubated with the respective secondary antibodies (1:1000) conjugated with horseradish peroxidase (HRP, Santa Cruz, Calif., USA) for 1 h at room temperature, as shown below in Table 12.

Characterization of Samples Treated with Culture Medium UL-MD-Versus Controls (CTRL 1-11)

The results relating to the expression of collagen type I, collagen type IV and of the cytokeratins (CK) 1, 5, 10 and 14 were expressed with a quantitative scale as follows:

TABLE 12

| | CTRL | CTRL | | | | | | | | CTRL | CTRL | Sa [symbol] |
|---|---|---|---|---|---|---|---|---|---|---|---|---|
| Markers | neg1 | pos2 | CTRL 3 | CTRL 4 | CTRL 5 | CTRL 6 | CTRL 7 | CTRL 8 | CTRL 9 | 10 | 11 | 12 |
| Collagen | −/+ | ++ | + | + | − | − | −/+ | −/+ | −/+ | −/+ | − | ++ [symbol] |
| type I | | | | | | | | | | | | |
| Collagen | −/+ | ++ | + | − | − | + | + | −/+ | ++ | + | −/+ | ++ [symbol] |
| type IV | | | | | | | | | | | | |
| CK-1 | + | +++ | + | ++ | + | + | −/+ | −/+ | −/+ | −/+ | + | ++ [symbol] |
| CK-5 | + | ++ | + | − | + | − | + | −/+ | + | + | −/+ | ++ [symbol] |
| CK-10 | −/+ | +++ | + | + | − | − | −/+ | + | +++ | −/+ | + | ++ [symbol] |
| CK-14 | ++ | ++ | ++ | + | + | + | + | −/+ | −/+ | −/+ | ++ | ++ [symbol] |

Legend
−−− = absence of band
−/+ = slight presence of band
+ = thin band present
++ = medium band present
+++ = broad band present
++++ = high band present
+++++ = diffuse band present [symbol]
indicates data missing or illegible when filed

### Fluorescence Activated Cell Sorting (FACS)

Cellular suspensions obtained after digestion with collagenase were transferred to 15-ml test tubes (Nunc, Kamstrup, Denmark) and fixed in 4% paraformaldehyde solution for 30 minutes at room temperature. After three washings with PBS (pH 7.4), the cells were permeabilized with Triton 0.1% for 15 minutes, 4% at room temperature. The pellets of the samples (Sample, 12) and of the controls (CTRLs, 1-11) were resuspended to a final concentration of 5×10⁵ cells/ml in PBS and incubated with the following primary antibodies (ABD serotec, Kidlington-Oxford, UK): anti-collagen type I, anti-collagen type anti-IV and anti-cytokeratins (CK)-1, -5, -10, -14. All monoclonal antibodies conjugated with R-phycoerythrin (PE) or fluorescein isothiocyanate (FITC) were used. Specific controls with the corresponding isotypes were divided for each monoclonal antibody (ABD serotec). The samples were submitted to quantitative analysis by a laser cytofluorometer (Epics profile II, Coulter, Hialeath, FL) at 488 nm, where the percentage of fluorescent cells (PFC) reflects the geometric mean of the field of interest. The threshold values (gates) were established using control samples labelled with the corresponding isotype. All the values were analysed with a minimum threshold of 15 000 cells.

FACS. Characterization in Absolute Values of Samples Treated with Culture Medium UL-MD Versus Controls

The results relating to the expression of collagen type I, collagen type IV and of cytokeratins (CK) 1, 5, 10 and 14 were expressed with a quantitative scale relative to the geometric mean+/−Standard Deviation (DS), as follows:

TABLE 13

| | CTRL | CTRL | | | | | |
|---|---|---|---|---|---|---|---|
| Markers | neg1 | pos2 | CTRL 3 | CTRL 4 | CTRL 5 | CTRL 6 | CTRL 7 |
| Collagen | 2 +/− 2 | 45 +/− 10 | 15 +/− 4 | 12 +/− 3 | 5 +/− 4- | 3 +/− 2 | 4 +/− 2 |
| type I | | | | | | | |
| Collagen | 3 +/− 2 | 40 +/− 5 | 18 +/− 3 | 9 +/− 8 | 7 +/− 6 | 12 +/− 8 | 10 +/− 7 |
| type IV | | | | | | | |
| CK-1 | 3 +/− 1 | 38 +/− 9 | 11 +/− 10 | 29 +/− 7 | 11 +/− 9 | 14 +/− 11 | 9 +/− 7 |
| CK-5 | 2 +/− 1 | 66 +/− 18 | 15 +/− 12 | 6 +/− 5 | 8 +/− 5 | 2 +/− 2 | 12 +/− 10 |
| CK-10 | 5 +/− 3 | 24 +/− 6 | 4 +/− 2 | 9 +/− 7 | 1 +/− 1 | 1 +/− 1 | 2 +/− 1 |
| CK-14 | 10 +/− 8 | 56 +/− 22 | 9 +/− 7 | 11 +/− 8 | 19 +/− 14 | 10 +/− 7 | 12 +/− 9 |

| | | | CTRL | CTRL | Sa [symbol] |
|---|---|---|---|---|---|
| Markers | CTRL 8 | CTRL 9 | 10 | 11 | 12 |
| Collagen | 6 +/− 4 | 7 +/− 5 | 5 +/− 3 | 7 +/− 6 | 222 +/− 28 |
| type I | | | | | |
| Collagen | 7 +/− 5 | 23 +/− 8 | 13 +/− 9 | 10 +/− 8 | 130 +/− 11 |
| type IV | | | | | |
| CK-1 | 5 +/− 3 | 3 +/− 2 | 17 +/− 15 | 18 +/− 14 | 299 +/− 31 |
| CK-5 | 3 +/− 1 | 18 +/− 12 | 1 +/− 1 | 3 +/− 1 | 189 +/− 18 |
| CK-10 | 4 +/− 1 | 78 +/− 30 | 22 +/− 20 | 19 +/− 16 | 200 +/− 48 |
| CK-14 | 2 +/− 1 | 2 +/− 1 | 2 +/− 1 | 42 +/− 12 | 39 +/− 11 |

[symbol]

indicates data missing or illegible when filed

FACS. Characterization in Percentage Value of Samples Treated with Culture Medium UL-MD Versus Controls

The results relating to the expression of collagen type I, collagen type IV and of cytokeratins (CK) 1, 5, 10 and 14 were expressed with a quantitative scale relative to the fluorescence expressed as a percentage value relative to the isotypes, after deducting the assumed nonspecific fluorescence (specific gate), as follows:

TABLE 14

| | CTRL | CTRL | | | | | | | | CTRL | CTRL | Sa [symbol] |
|---|---|---|---|---|---|---|---|---|---|---|---|---|
| Markers | neg1 | pos2 | CTRL 3 | CTRL 4 | CTRL 5 | CTRL 6 | CTRL 7 | CTRL 8 | CTRL 9 | 10 | 11 | 12 |
| Collagen | 0.2% | 3% | 5% | 3.9% | 0.5% | 0.3% | 0.9% | 0.9% | 1% | 0.5% | 0% | 81. [symbol] |
| type I | | | | | | | | | | | | |
| Collagen | 0.3% | 5% | 7% | 0.3% | 0.1% | 1.5% | 1.9% | 0.9% | 8% | 3% | 1% | 58 [symbol] |
| type IV | | | | | | | | | | | | |
| CK-1 | 0.3% | 7% | 1% | 14% | 2% | 3% | 1.8% | 1% | 0.9% | 3% | 3.3% | 98 [symbol] |
| CK-5 | 0.2% | 12% | 1.5% | 0% | 1.2% | 0% | 0.7% | 1% | 1.8% | 0.1% | 0.7% | 70. [symbol] |
| CK-10 | 0.5% | 7.6 | 1% | 1.9% | 0% | 0% | 0.3% | 0.5% | 10% | 2% | 0.9% | 78 [symbol] |
| CK-14 | 1% | 13% | 1% | 1.25% | 2% | 1.7% | 0.8% | 0.1% | 0.1% | 0.1% | 17% | 20. [symbol] |

[symbol]

indicates data missing or illegible when filed

### Example 2

### Clinical Studies In Vivo

### Formulations UL-MD, UL-PI and RITROFIX.

### Clinical Study In Vivo 1.

A clinical study was conducted for assessing the tolerability and therapeutic efficacy, in regeneration and repair of the dermis and hypodermis, of the products designated UL-MD, UL-PI and RITROFIX (composition given in Tables 1, 2 and 3).

The present study was conducted on mammals of a sample constituted of dogs, cats (of various breeds and sizes) and humans, these too of various ages and races, presenting with cutaneous lesions ascribable to tissue atrophy (Canidae: resulting from laceration and contusion wounds, resulting from surgical wound, resulting from wound from licking and resulting from burns).

Subjects with the lesions stated above were selected at the inclusion visit.

The animals were brought to the clinic for a weekly check-up until recovery and underwent cytological examination and assessment of the extent and depth of the cutaneous lesion.

### Case 1

Dog, miniature poodle, 10 years, female, was brought to the clinic because of an ulcerous lesion of the foot and self-inflicted lesions from licking on the skin. After correction by escharotomy there were still reactive atrophic cutaneous lesions as a result of chronic licking.

Clinical Examination.

Erythema, cutaneous ulcers following fibrosis of the dorsal portion of the metacarpus and of the phalanges.

Diagnosis.

Clinical picture attributable to chronic self-inflicted dermatitis complicated by superficial secondary infections. Cytological examination identified a bacterial infection of the ulcerated lesion.

Therapy.

Systemic antibiotic therapy with enrofloxacin 5 mg/kg per os was administered for three weeks. The wound was cleaned with iodine-povidone on the first day, followed by cleaning of the wound with saline and application of the preparation RITROFIX twice daily for 30 days.

Check-Up Visits.

After a week there was improvement of the infection and 80% regeneration of tissue. After two weeks, disappearance of infection was observed, and further reduction of the lesions.

Clinical Outcome.

At subsequent examination after four weeks from the start of treatment, complete disappearance of dermal fibrosis was observed.

### Case 2

Chartreux cat, female, 12 years old, brought to the clinic because of wounds resulting from multiple dorsocaudal burns.

Clinical Examination.

Map-like dorsocaudal lesions with exudation and formation of atrophic-purulent-fibrotic scars incorporating hairs.

Diagnosis.

Cytologic examination showed bacterial superinfection of the burn wound with atrophic scarring.

Therapy.

Marbofloxacin was administered per os at 2.5 mg/kg for 21 days. The fur was shaved and the wound was cleaned with iodine-povidone iodate followed by application of the preparation UL-PI twice daily for 60 days.

Check-Up Visits.

After a week, granulation tissue was observed with about 40% reduction in size of the lesions and at the end of the second week the wound had healed completely, with formation of stable regenerated tissue.

Clinical Outcome.

At the subsequent check-up after 60 from the start of treatment, all the scars appeared whitish in colour, of normal resistance and consistency without a fibrous component.

### Case 3

Dog, Golden retriever, female 7 years.

Anamnesis.

Cutaneous lacerations left forelimb third distal, secondary to road injury resulting in atrophic secondary ulcer. Following numerous attempts at surgical correction, the patient had undergone numerous treatments: tetracyclines combined with deposited corticosteroid, local disinfection in the areas of the lesions with aminosidine sulphate+prednisolone and clostebol acetate: no noteworthy result and cutaneous hyperextensibility induced. Multiple skin biopsies were performed, showing alteration of the collagen fibres consistent with Ehlers-Danlos syndrome.

Day 0.

The wounds and cutaneous lacerations were treated with polyvinyl-pyrrolidone twice daily and preparation UL-MD with occlusive bandage on the limbs and free application, without bandages, in the ischiatic area.

This was combined with systemic antibiotic therapy with amoxicillin and clavulanic acid 12.5 mg/kg Bid for controlling the secondary infections.

Day 30. After 30 days, re-epithelialization of all the wounds treated with the preparation UL-MD was observed, and these scars did not exhibit atrophy.

Use of the preparation UL-MD resulted in eutrophic healing, within just 30 days, of extensive cutaneous wounds which for months had been complicated by superinfections and atrophic scarring, owing to genetic collagen disease present since birth.

### Case 4

Description.

Dog WHWT, male 10 years of age.

Anamnesis.

For about six months the dog had ulcerous cutaneous interdigital areas on the forelimbs. Surgical debridement was followed by antibiotic therapy with cephalexin 20 mg/kg/bid for 15 days.

Day 0.

After disinfection with iodine-povidone, the preparation RITROFIX was applied to the infradigital area once daily with occlusive bandage until the check-up.

Systemic antibiotic therapy was continued with cephalexin at the same doses.

Day 15.

Remission of the lesions was noted at the check-up visit.

Day 90.

Application of the preparation RITROFIX-CREAM led to eutrophic re-epithelialization of the surgically treated areas of the skin.

Day 180.

Application of the preparation RITROFIX led to healing of the surgical wound without relapse.

### Case 5

Woman, Caucasian, 75 years of age.

### Anamnesis and Therapeutic Regimen

The patient had been brought to the clinic owing to the presence of vascular stasis ulcers of the lower limbs that had been present for 10 years in the tibiotarsal region, with size of about 5 cm. Topical therapy was employed, with application of the preparation UL-MD twice daily for 60 days.

Day 0.

The atrophic lesions in the tibial area were symmetrical on both lower limbs of about 5 cm and with moderate perilesional erythema.

Day 60.

At check-up, the lesions had diminished by 90% relative to the start of therapy.

### Summary of the Clinical Progression

In this case there was a gradual reduction in diameter of the ulcers and at 90 days there was complete re-epithelialization of them.

### Case 6

Man, Caucasian, 88 years of age.

### Anamnesis and Therapeutic Regimen

The patient had a lateral bedsore that had been present for about 7 days, which had been treated solely with 10% iodine povidone. At the time of inclusion (day 0) the preparation UL-PI was applied twice daily for 15 days.

Day 0.

Presence of erosions, ulcers and perilesional depigmentation with loss of substance of approx. 7 cm.

Day 7.

The serocellular crust present at the time of examination had regressed and the depth of the lesion had decreased by 50%.

Day 14.

Retrophization and re-epithelialization of the sore were observed at the final visit, and flattening of the margins with a rapid, progressive re-pigmentation of the skin.

### Case 7

Woman, Caucasian, 69 years of age.

### Anamnesis and Therapeutic Regimen

The patient had the results of secondary atrophy that occurred on the scar from a cut on the left lower limb in the anterior zone, to the right of the tibial crest. Topical therapy was employed, with application of the preparation RITROFIX twice daily for 60 days.

Day 0.

The lesion was of about 3 cm, atrophic, purulent and with slight perilesional erythema.

Day 60.

At check-up the lesion had decreased 100% relative to the start of therapy.

### Case 8

Woman, Caucasian, 70 years of age.

### Anamnesis and Therapeutic Regimen

The patient had the results of secondary atrophy that occurred on the scar from exeresis of a junction naevus in the popliteal fossa. Topical therapy was employed with application of the preparation RITROFIX twice daily for 60 days.

Day 0.

The lesion in the area in question was of about 1 cm, hypoplastic and depigmented.

Day 60.

At check-up the lesion had decreased by 100% relative to the start of therapy.

### Clinical Study In Vivo 2.

The composition RITROFIX was used on 40 subjects on a compassionate basis, who had:
A) ulcers resulting from traumatic lesions;
B) ulcers resulting from venous stasis.

The most significant data are summarized below in Table 15.

The lesions affected the limbs almost exclusively, with a clear prevalence of the lower limbs.

TABLE 15

| Subjects treated | Number | male | female | Average age |
|---|---|---|---|---|
| Group A | 20 | 10 | 10 | 68 +/− 3 |
| Group B | 20 | 10 | 10 | 82 +/− 3 |
| Total | 40 | 20 | 20 | 75 +/− 10 |

The composition RITROFIX was used in two daily applications for a period of not less than 35 days up to a maximum of 90 days. There were no cases of development of phenomena of allergy or intolerance, in fact mostly after a few days from the start of treatment the patients reported attenuation of pruritus, pain and a feeling of local tightness. Initially, in nearly all subjects, hyperaemia was found, as an effect of superficial vasodilatation induced by the composition RITROFIX; this is undoubtedly a positive effect considering the state of occlusion of many vessels found in the perilesional zone. The initial hyperaemia was often accompanied by an increase in local pigmentation through the favourable action of zinc oxide on melanin biosynthesis. However, the hyperaemia and hyperpigmentation gradually became attenuated as the treatment continued. In the majority of subjects there was total or almost total regression of the lesions (81%). Good results were found for the majority of atrophic stasis ulcers (98%), normalization of colouring and reduction in size. In chronic post-traumatic lesions there was a significant reduction thereof and recovery of substance (80%) together with macroscopic changes such as variation of colouring, attenuation of the fibrous shoots, and spontaneous re-epithelialization.

Moreover, the composition RITROFIX in total and its individual constituents were used blind on 180 subjects on a compassionate basis for a maximum of 90 days, who had:

### Group A

A) ulcers resulting from traumatic lesions treated with the composition RITROFIX, as described in Table 3;

A-bis) ulcers resulting from traumatic lesions treated only with the adamantane derivative constituent, as described in Table 4;

A-tris) ulcers resulting from traumatic lesions treated only with the proteolytic enzyme constituent, as described in Table 5;

A-quater) ulcers resulting from traumatic lesions treated only with the salicylate constituent, as described in Table 6;

A-quinquies) ulcers resulting from traumatic lesions treated only with the amino acid constituent, as described in Table 7;

A-sexies) ulcers resulting from traumatic lesions treated only with the vitamin constituent, as described in Table 8;

A-septies) ulcers resulting from traumatic lesions treated only with the essential oil constituent, as described in Table 9;

A-octies) ulcers resulting from traumatic lesions treated only with the essential oil constituent, as described in Table 10;

A-nonies) ulcers resulting from traumatic lesions treated only with the essential oil constituent, as described in Table 11.

### Group B

B) ulcers resulting from venous stasis treated with the composition RITROFIX, as described in Table 3;

B-bis) ulcers resulting from venous stasis treated only with the adamantane derivative constituent, as described in Table 4;

B-tris) ulcers resulting from venous stasis treated only with the proteolytic enzyme constituent, as described in Table 5;

B-quater) ulcers resulting from venous stasis treated only with the salicylate constituent, as described in Table 6;

B-quinquies) ulcers resulting from venous stasis treated only with the amino acid constituent, as described in Table 7;

B-sexies) ulcers resulting from venous stasis treated only with the vitamin constituent, as described in Table 8;

B-septies) ulcers resulting from venous stasis treated only with the essential oil constituent, as described in Table 9;

B-octies) ulcers resulting from venous stasis treated only with the essential oil constituent, as described in Table 10;

B-nonies) ulcers resulting from venous stasis treated only with the essential oil constituent, as described in Table 11.

The most significant data are summarized below in Table 16.

The lesions always relate to the lower limbs.

TABLE 16

| | | | | Average |
|---|---|---|---|---|
| Subjects treated | N. | Male (M) | Female (F) | age |
| Group A | 90 | 45 | 45 | 68 +/− 8 |
| Group B | 90 | 45 | 45 | 76 +/− 8 |
| Total | 180 | 90 | 90 | 72 +/− 12 |

| Groups | N. | M | F | lesion |
|---|---|---|---|---|
| A) | 10 | 5 | 5 | complete re-epithelialization on |
| | | | | average at day 30 of treatment |
| | | | | (98% of those treated) |
| A-bis) | 10 | 5 | 5 | no re-epithelialization, extensive |
| | | | | hyperaemia (56% of those |
| | | | | treated) |
| A-ter) | 10 | 5 | 5 | no re-epithelialization, mild |
| | | | | hyperaemia (68% of those |
| | | | | treated) |
| A-quater) | 10 | 5 | 5 | no re-epithelialization, mild |
| | | | | hyperaemia and |
| | | | | hyperpigmentation (62% of those |
| | | | | treated) |
| A-quinquies) | 10 | 5 | 5 | no re-epithelialization, |
| | | | | hyperkeratosis (12% of those |
| | | | | treated) |
| A-sexies) | 10 | 5 | 5 | no re-epithelialization, |
| | | | | hyperpigmentation (38% of those |
| | | | | treated) |
| A-septies) | 10 | 5 | 5 | no re-epithelialization, mild |
| | | | | hyperaemia (32% of those |
| | | | | treated) |
| A-octies) | 10 | 5 | 5 | no re-epithelialization, |
| | | | | suppuration (28% of those |
| | | | | treated) |
| A-nonies) | 10 | 5 | 5 | no re-epithelialization, mild |
| | | | | hyperaemia (40% of those |
| | | | | treated) |
| Total Group A | 90 | 45 | 45 | Note: re-epithelialization of the |
| | | | | lesion only in group A) treated |
| | | | | with the complete composition |
| | | | | RITROFIX |
| B) | 10 | 5 | 5 | complete re-epithelialization on |
| | | | | average at day 30 of treatment |
| | | | | (80% of those treated) |
| B-bis) | 10 | 5 | 5 | no re-epithelialization, extensive |
| | | | | hyperaemia (48% of those |
| | | | | treated) |
| B-ter) | 10 | 5 | 5 | no re-epithelialization, mild |
| | | | | hyperaemia (70% of those |
| | | | | treated) |
| B-quater) | 10 | 5 | 5 | no re-epithelialization, mild |
| | | | | hyperaemia and |
| | | | | hyperpigmentation (42% of those |
| | | | | treated) |
| B-quinquies) | 10 | 5 | 5 | no re-epithelialization, |
| | | | | hyperkeratosis (30% of those |
| | | | | treated) |
| B-sexies) | 10 | 5 | 5 | no re-epithelialization, |
| | | | | hyperpigmentation (18% of those |
| | | | | treated) |
| B-septies) | 10 | 5 | 5 | no re-epithelialization, mild |
| | | | | hyperaemia (12% of those |
| | | | | treated) |
| B-octies) | 10 | 5 | 5 | no re-epithelialization, |
| | | | | suppuration (35% of those |
| | | | | treated) |
| B-nonies) | 10 | 5 | 5 | no re-epithelialization, pruritus |
| | | | | (20% of those treated) |
| Total Group B | 90 | 45 | 45 | Note: re-epithelialization of the |
| | | | | lesion only in group B) treated |
| | | | | with the complete composition |
| | | | | RITROFIX |
| Total Groups A + B | 180 | 90 | 90 | Note: re-epithelialization of the |
| | | | | lesion only in groups A) and B) |
| | | | | treated with the complete |
| | | | | composition RITROFIX |

The composition RITROFIX was used in two daily applications for a period of not less than 35 days up to a maximum of 90 days, versus its individual constituents. There were no cases of development of phenomena of allergy or intolerance, in fact mostly after a few days from the start of treatment the patients reported attenuation of pruritus, pain and a feeling of local tightness. Initially, in nearly all subjects, superficial hyperaemia was found in the perilesional zone. The initial hyperaemia was accompanied in some cases by an increase of local pigmentation. However, as the treatment continued, the hyperaemia and hyperpigmentation were attenuated progressively only in the groups treated with the complete RITROFIX composition. In the majority of subjects treated with the RITROFIX complete formulation there was total or almost total regression of the lesions (atrophic stasis ulcers in 98% of the subjects treated; chronic post-traumatic lesions in 80% of the subjects treated) together with gradual spontaneous re-epithelialization. In contrast, in all the subgroups treated with just one of the constituents of the composition under investigation, there was never re-epithelialization of the damaged tissues. This phenomenon demonstrates how the synergism and potentiation of action, resulting from the combination of constituents, establish a novel composition endowed with significant and innovative regenerative efficacy on tissues affected by atrophic-degenerative lesions.

1. A composition comprising as an active ingredient a salt of an adamantane carboxylic acid in combination with at least one further active ingredient selected from proteolytic enzymes, willow extract, salicylic acid, acetylsalicylic acid, salicylates and acetylsalicylates.

2. The composition according to claim 1, comprising a salt of an adamantane carboxylic acid, a proteolytic enzyme and a further active ingredient selected from willow extract, salicylic acid, acetylsalicylic acid, salicylates and acetylsalicylates.

3. The composition according to claim 1, further comprising zinc oxide.

4. The composition according to claim 1, comprising at least one further active ingredient selected from the group consisting of vitamins, phytoextracts, sugars, amino acids, further salts and mineral salts, ventilated green clay, and any combination thereof.

5. The composition according to claim 1, wherein the salt of an adamantane carboxylic acid is a salt with an adamantane base and/or aromatic base.

6. The composition according to claim 1, wherein the salt of an adamantane carboxylic acid has the following general formula: 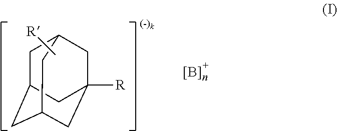
in which:
k=1 or 2;
n=1 or 2;
R is selected from —C(O)O⁻ and -Ak-C(O)O⁻, in which Ak is optionally substituted C₁₋₆ alkyl;
R′ is selected from hydrogen, —C(O)O⁻ and -Ak-C(O)O⁻, wherein Ak is optionally substituted C₁₋₆ alkyl; and
B is a counter-ion selected n times independently of one another from the group consisting of the bases of formulae (i) and (ii): 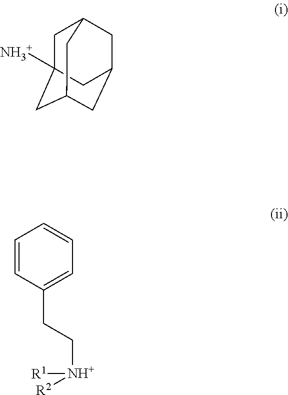
in which R¹ and R² are selected independently of one another from hydrogen and C₁₋₆ alkyl.

7. The composition according to claim 6, wherein the salt of an adamantane carboxylic acid is selected from 1-adamantane acetate of N,N-dimethyl-phenylethylammonium and 1,3-adamantane dicarboxylate of bis(1-adamantane ammonium), having the following structural formulae, respectively:

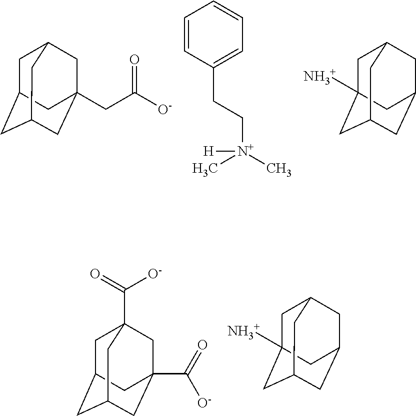 [SMILES: O=C([O-])CC12CC3CC(CC(C3)C1)C2 ; C[NH+](C)CCc1ccccc1 ; [NH3+]C12CC3CC(CC(C3)C1)C2 ; O=C([O-])C12CC3CC(C1)CC(C(=O)[O-])(C3)C2 ; [NH3+]C12CC3CC(CC(C3)C1)C2]

8. The composition according to claim 1, wherein the proteolytic enzyme is selected from the group consisting of papain, collagenase, serratiopeptidase, heparanase, elastase, bromelain, bradykinase, Clostridium peptidase, proteolytic enzymes expressed by Lactobacillus acidophilus, proteolytic enzymes expressed by the genus Aspergillus, protease, alliinase and fibrinolysin.

9. The composition according to claim 1, wherein the vitamins are selected from the group consisting of retinoic acid, retinaldehyde, retinol, alpha-tocopherol, beta-carotene, colecalciferol, ascorbic acid, pantothenic acid, dexpanthenol, D-calcium pantothenate, cocarboxylase tetrahydrate, pyridoxine, pyridoxine-HCl, folic acid, niacinamide, nicotinamide, riboflavin, riboflavin dihydrate sodium phosphate, cyanocobalamin, para-aminobenzoic acid, biotin and any combination thereof.

10. The composition according to claim 1, wherein the phytoextract is selected from the group consisting of glycyrrhizin, glycyrrhetic acid, liquorice extracts, aloe extracts, aloe vera extracts, pineapple extracts, mallow extracts, extracts of St John's Wort, burdock extracts, lime extracts, borage extracts, clover extracts, mint extracts, juniper extracts, lavender extracts, marigold extracts, extracts of horse chestnut, grapefruit extracts, cedar extracts, myrtle extracts, pine extracts.

11. The composition according to claim 1, comprising human or veterinary medicament for the treatment of an atrophic tissue selected from cutaneous tissue, subcutaneous tissue, skin appendages, mucous tissue and/or connective tissue.

12. The composition according to claim 11, comprising a human or veterinary medicament for the treatment of ulcers and/or bedsores and/or necrotic tissue.

13. The composition according to claim 11 comprising a lyophilized, cream, gel, foam or injectable form.

14. A culture medium for cells or cutaneous, subcutaneous or mucous tissue in vitro, comprising a composition according to claim 1.

15. A method for the anti-atrophic and/or reparative-regenerative treatment of the skin and/or of the integumentary system, comprising applying a composition according to claim 1 to the skin and/or to the integumentary system.

16. The method according to claim 15, wherein the composition is in a lyophilized, cream, gel, foam or injectable form.
